(19) United States
(12) Patent Application Publication
(10) Pub. No.: US 2019/0209567 A1
(43) Pub. Date: Jul. 11, 2019

(54) A Specific Trifluoroethyl Quinoline Analogue For Use In The Treatment of APDS

(71) Applicant: UCB Biopharma SPRL, Brussels (BE)

(72) Inventors: Rodger Anthony Allen, Slough, Berkshire (GB); Martin John Armstrong, Brussels (BE); Marina Cavazzana, Paris (FR); Sven Kracker, Paris (FR); Duncan Philip McHale, Slough, Berkshire (GB); Andrew Charles Payne, Slough, Berkshire (GB)

(21) Appl. No.: 16/099,537

(22) PCT Filed: May 15, 2017
(86) PCT No.: PCT/EP2017/061567

§ 371 Date: Nov. 7, 2018

(30) Foreign Application Priority Data
May 19, 2016 (GB) 1608797.5

(51) Int. Cl. A61K 31/519; A61P 37/06

(52) U.S. Cl. CPC A61K 31/519; A61P 37/06

(57) ABSTRACT

N—{(R)-1-[8-Chloro-2-(1-oxypyridin-3-yl)-quinolin-3-yl]-2,2,2-trifluoroethyl}-pyrido[3,2-d]pyrimidin-4-ylamine is effective in the treatment and/or prevention of activated phosphoinositide 3-kinase delta syndrome (APDS).

The present invention relates to the new therapeutic use of a known chemical compound. More particularly, the present invention concerns the use of a specific substituted quinoline derivative comprising a fluorinated ethyl side-chain in the treatment of activated phosphoinositide 3-kinase delta syndrome (APDS).

N—{(R)-1-[8-Chloro-2-(1-oxypyridin-3-yl)quinolin-3-yl]-2,2,2-trifluoroethyl}-pyrido[3,2-d]pyrimidin-4-ylamine is specifically disclosed in WO 2012/032334. The compounds described in that publication are stated to be of benefit as pharmaceutical agents, especially in the treatment of adverse inflammatory, autoimmune, cardiovascular, neurodegenerative, metabolic, oncological, nociceptive and ophthalmic conditions.

There is no specific disclosure or suggestion in WO 2012/032334, however, that the compounds described therein might be beneficial in the treatment of APDS.

Activated phosphoinositide 3-kinase delta syndrome (APDS), also known as PASLI (p110δ-activating mutation causing senescent T cells, lymphadenopathy and immunodeficiency), is a serious medical condition that impairs the immune system. APDS patients generally have reduced numbers of white blood cells (lymphopenia), especially B cells and T cells, compromising their propensity to recognise and attack invading microorganisms, such as viruses and bacteria, and thereby prevent infection. Individuals affected with APDS develop recurrent infections, particularly in the lungs, sinuses and ears. Recurrent respiratory tract infections may gradually lead to bronchiectasis, a condition which damages the passages leading from the windpipe to the lungs (bronchi) and can cause breathing problems. APDS patients may also suffer from chronic active viral infections, including Epstein-Barr virus infections and cytomegalovirus infections.

APDS has also been associated with abnormal clumping of white blood cells, which can lead to enlarged lymph nodes (lymphadenopathy). Alternatively, the white blood cells can build up to form solid masses (nodular lymphoid hyperplasia), usually in the moist lining of the airways or intestines. Whilst lymphadenopathy and nodular lymphoid hyperplasia are benign (noncancerous), APDS also increases the risk of developing a form of cancer called B cell lymphoma.

APDS is a disorder of childhood, typically arising soon after birth. However, the precise prevalence of APDS is currently unknown.

Phosphoinositide 3-kinase delta (PI3Kδ) is a lipid kinase which catalyses the generation of phosphatidylinositol 3,4,5-trisphosphate (PIP3) from phosphatidylinositol 4,5-bisphosphate (PIP2). PI3Kδ activates signalling pathways within cells, and is specifically found in white blood cells, including B cells and T cells. PI3Kδ signalling is involved in the growth and division (proliferation) of white blood cells, and it helps direct B cells and T cells to mature (differentiate) into different types, each of which has a distinct function in the immune system.

APDS is known to occur in two variants, categorised as APDS1 and APDS2. APDS1 is associated with a heterozygous gain-of-function mutation in the PIK3CD gene encoding the PI3Kδ protein; whereas APDS2 is associated with loss-of-function frameshift mutations in the regulatory PIK3R1 gene encoding the p85α regulatory subunit of class I phosphoinositide 3-kinase (PI3K) peptides. Both mutations lead to hyperactivated PI3K signalling. See I. Angulo et al., Science, 2013, 342, 866-871; C. L. Lucas et al., Nature Immunol., 2014, 15, 88-97; and M-C. Deau et al., J. Clin. Invest., 2014, 124, 3923-3928.

There is currently no effective treatment available for APDS. Because of the seriousness of the condition, and the fact that it arises in infancy, the provision of an effective treatment for APDS would plainly be a highly desirable objective.

It has now been found that N—{(R)-1-[8-chloro-2-(1-oxypyridin-3-yl)quinolin-3-yl]-2,2,2-trifluoroethyl}pyrido[3,2-d]pyrimidin-4-ylamine is capable of inhibiting the elevation of PI3K signalling in T cells (lymphocytes) from both APDS1 and APDS2 patients in the presence or absence of T cell receptor activation.

The present invention accordingly provides N—{(R)-1-[8-chloro-2-(1-oxypyridin-3-yl)quinolin-3-yl]-2,2,2-trifluoroethyl}pyrido[3,2-d]pyrimidin-4-ylamine of formula (A):

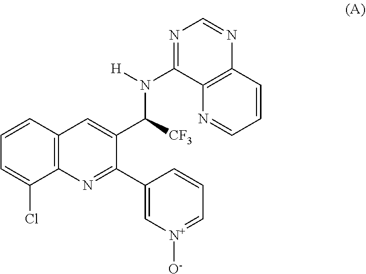 [SMILES: [O-][n+]1cccc(-c2nc3c(Cl)cccc3cc2[C@@H](Nc2ncnc3cccnc23)C(F)(F)F)c1]

or a pharmaceutically acceptable salt thereof, for use in the treatment and/or prevention of APDS.

The present invention also provides a method for the treatment and/or prevention of APDS, which method comprises administering to a patient in need of such treatment an effective amount of N—{(R)-1-[8-chloro-2-(1-oxypyridin-3-yl)quinolin-3-yl]-2,2,2-trifluoroethyl}pyrido[3,2-d]pyrimidin-4-ylamine of formula (A) as depicted above, or a pharmaceutically acceptable salt thereof.

The present invention also provides the use of N—{(R)-1-[8-chloro-2-(1-oxypyridin-3-yl)quinolin-3-yl]-2,2,2-trifluoroethyl}pyrido[3,2-d]pyrimidin-4-ylamine of formula (A) as depicted above, or a pharmaceutically acceptable salt thereof, for the manufacture of a medicament for the treatment and/or prevention of APDS.

For the effective treatment and/or prevention of APDS, a pharmaceutical composition may be provided which comprises N—{(R)-1-[8-chloro-2-(1-oxypyridin-3-yl)quinolin-3-yl]-2,2,2-trifluoroethyl}pyrido[3,2-d]pyrimidin-4-ylamine of formula (A) as depicted above, or a pharmaceutically acceptable salt thereof, in association with a pharmaceutical carrier. Typical pharmaceutical compositions may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges, capsules, solutions, syrups or suspensions, or they may be presented as a dry product for constitution with water or other suitable vehicle before use. For buccal administration, the compositions may take the form of tablets or lozenges. For parenteral administration, the compositions may be formulated for injection, e.g. by bolus injection or infusion, for subcutaneous administration, or as a long-acting formulation, e.g. a depot preparation which may be administered by implantation or by intramuscular injection; formulations for injection may be presented in unit dosage form, e.g. in glass ampoules or multi-dose containers, e.g. glass vials, and may take such forms as suspensions, solutions or emulsions in oily or aqueous vehicles, or the active ingredient may be in powder form for constitution with a suitable vehicle, e.g. sterile pyrogen-free water, before use. For nasal administration or administration by inhalation, the composition may take the form of an aerosol spray presentation for pressurised packs or a nebuliser. For topical administration, the composition may take the form of an ointment or lotion. For ophthalmic administration the composition may be formulated as a micronized suspension or an ointment. For rectal administration, the compositions may be formulated as suppositories.

The compositions may be formulated by conventional methods well known in the pharmaceutical art, for example as described in Remington: the Science and Practice of Pharmacy, Pharmaceutical Press, 22^{nd} Edition, 2012.

For use in the treatment and/or prevention of APDS, N—{(R)-1-[8-chloro-2-(1-oxypyridin-3-yl)quinolin-3-yl]-2,2,2-trifluoroethyl}pyrido[3,2-d]pyrimidin-4-ylamine, or a pharmaceutically acceptable salt thereof, may suitably be administered at a daily dosage of about 1 ng/kg to 1000 mg/kg, generally about 2 ng/kg to 500 mg/kg, typically about 5 ng/kg to 200 mg/kg, appositely about 10 ng/kg to 100 mg/kg, ideally about 10 ng/kg to 50 mg/kg, more particularly about 10 ng/kg to 40 mg/kg, of body weight. The active ingredient will typically be administered on a regimen of 1 to 4 times a day.

If desired, N—{(R)-1-[8-chloro-2-(1-oxypyridin-3-yl)quinolin-3-yl]-2,2,2-trifluoroethyl}pyrido[3,2-d]pyrimidin-4-ylamine, or a pharmaceutically acceptable salt thereof, may be co-administered with another pharmaceutically active agent, e.g. an anti-inflammatory molecule such as methotrexate or hydroxychloroquine.

Specific aspects of the invention will now be described.

The activity of N—{(R)-1-[8-Chloro-2-(1-oxypyridin-3-yl)quinolin-3-yl]-2,2,2-trifluoroethyl}pyrido[3,2-d]pyrimidin-4-ylamine [hereinafter referred to as “Compound (A)”] on PI3K signalling was assessed by measuring levels of either phosphorylated serine 473 of AKT (pAKT^{S473}) or phosphorylated serine 235/236 of ribosomal protein S6 (pS6^{S235/236}) using flow cytometry. The results obtained are depicted in the accompanying drawings, in which:

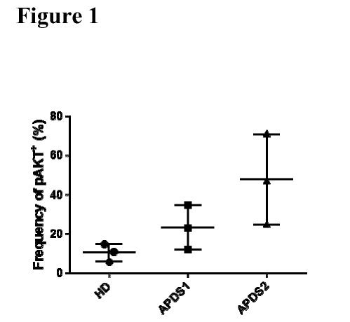

FIG. 1 shows the basal expression of pAKT^{S473} in peripheral T cell lymphoblasts derived from healthy donors (HD) (●), from APDS1 patients (▪), and from APDS2 patients (▴), by proportion of pAKT positive cells. Mean values±SD (standard deviation) are indicated.

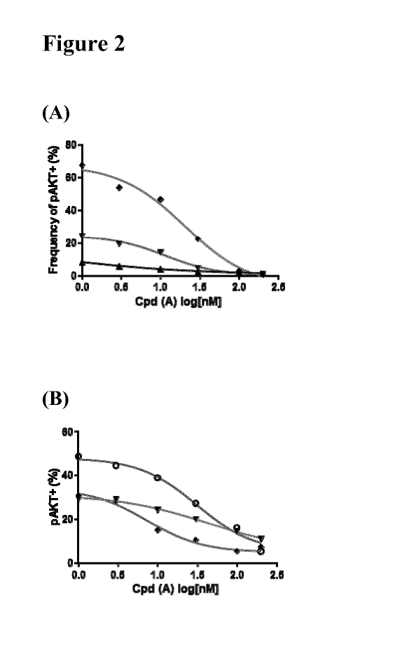

FIG. 2(A) displays representative data showing the effect of concentration responses of Compound (A) on the expression of pAKT^{S473} in T cell lymphoblasts from healthy donors (CTRL 1) (▴), from an APDS1 patient (CD_4) (▾), and from an APDS2 patient (R1_2) (♦), in the absence of T cell activation by OKT3. FIG. 2(B) displays representative data showing the effect of concentration responses of Compound (A) on the expression of pAKT^{S473} in T cell lymphoblasts from healthy donors (CTRL 1) (▾), from an APDS1 patient (CD_4) (♦), and from an APDS2 patient (R1_2) (◯), in the presence of T cell activation by OKT3. The expression of pAKT^{S473} was determined by flow cytometry.

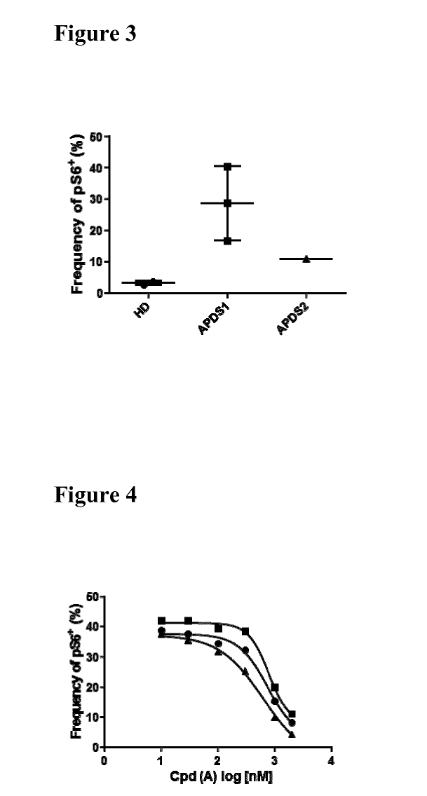

FIG. 3 shows the expression of pS6^{S235/236} in CD3⁺ cells from healthy donors (HD) (●), from APDS1 patients (▪), and from APDS2 patients (▴), by proportion of pS6^{S235/236} positive cells. Mean values±SD are indicated.

FIG. 4 displays representative data showing the effect of concentration responses of Compound (A) (concentration not adjusted for protein binding) on the expression of pS6^{S235/236} in T cell subsets (● CD3+; ▪ CD8+; ▴ CD4+) in whole blood from an APDS1 patient (CD_4). Expression of pS6^{S235/236} was determined by flow cytometry.

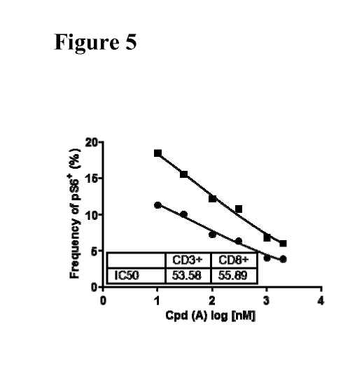

FIG. 5 displays representative data, plotted by the frequency of pS6⁺ cells, showing the effect of concentration responses of Compound (A) (concentration not adjusted for protein binding) on the expression of pS6^{S235/236} in T cell subsets (● CD3+; ▴ CD8+) in whole blood from an APDS2 patient (R1_4). The inset tables are the IC50 values (nM). Expression of pS6^{S235/236} was determined by flow cytometry.

### EXAMPLE 1: IN VITRO ANALYSIS OF PI3K SIGNALLING IN T CELL LYMPHOBLASTS

### Method

Analysis of phosphorylated AKT at Ser 473 (pAKT^{S473}) levels in T cell lymphoblast cultures by flow cytometry was performed with peripheral blood lymphocytes from healthy donors, and from APDS1 and APDS2 patients, with and without T cell receptor activation.

Generation of T cell lymphoblasts was performed in accordance with the method described by M-C. Deau et al. in J. Clin. Invest., 2014, 124, 3923-3928. In brief, peripheral blood mononuclear cells were isolated by Ficoll-Paque density gradient centrifugation (Pharmacia Biotech; catalogue no. #171-44003) and washed twice with RPMI 1640 GlutaMax medium (Invitrogen). T cell lymphoblasts were obtained by stimulating 1×106 cells per mL in RPMI 1640 GlutaMax medium supplemented with 10% human AB serum, penicillin/streptomycin (Invitrogen), PMA (phorbol 12-myristate 13-acetate; 20 ng/mL; Sigma-Aldrich) and ionomycin (1 μmol/L). After 2 to 3 days of activation, viable cells were separated by Ficoll-Paque density-gradient centrifugation and washed twice with RPMI 1640 GlutaMax medium, then cultured in RPMI 1640 GlutaMax medium supplemented with 10% human AB serum and 100 U/mL pro-IL2.

Analysis of phosphorylated AKT at Ser 473 levels in T cell lymphoblast cultures was performed once sufficient cells were available for the analysis (e.g. 6-12 days after starting the culture with pro-IL2).

The activity of Compound (A) was assessed in (i) the absence or (ii) the presence of T cell activation by receptor cross-linking with OKT3:

(i) Incubation of APDS1 and APDS2 patient cells for 30 minutes with Compound (A) at different concentrations (0, 1, 3, 10, 30, 100 and 200 nM). A positive control for pAKT staining (simulated cells with OKT3) was included (8×10⁶ cells/patient).

(ii) Incubation of APDS1 and APDS2 patient cells for 30 minutes with Compound (A) at different concentrations (0, 1, 3, 10, 30, 100 and 200 nM) stimulated with anti-CD3 (OKT3; T cell receptor activation).

Corresponding assays were performed with T cell lymphoblasts derived from healthy donors (CTRL).

### Results

### PI3K Signalling in T Cell Lymphoblasts

The level of PI3K signalling in T cell lymphoblasts from healthy donors, and from APDS1 and APDS2 patients, was assessed by measurement of pAKT^{S473} by flow cytometry. The results are displayed in FIG. 1. As can be seen from FIG. 1, the levels of PI3K signalling were elevated both in APDS1 and in APDS2 patient cells compared to healthy individuals.

### Effect of Compound (A) on PI3K Signalling in Unstimulated and OKT3-Stimulated T Cell Lymphoblasts

The effect of Compound (A) on expression of phosphorylated AKT at Ser 473 (pAKT^{S473}) was determined by flow cytometry in T cell lymphoblast cultures performed with peripheral blood lymphocytes from three (3) healthy donors, three (3) APDS1 patients and three (3) APDS2 patients, with and without T cell receptor activation by OKT3. The results are displayed in FIG. 2.

The IC50 values of Compound (A) for inhibition of pAKT^{S473} expression in T lymphoblasts derived from three (3) healthy donors, from three (3) APDS1 patients and from three (3) APDS2 patients, in the presence (+) and absence (−) of T cell activation by OKT3, are summarised in Table 1:

| | | | | | Healthy |
|---|---|---|---|---|---|
| | APDS1 | | APDS2 | | donors |
| OKT3 | + | − | + | − | + |
| Geomean IC50 (nM) | 21 | 8 | 28 | 13 | 16 |
| Range (nM) | 7-50 | 3-12 | 21-33 | 8-20 | 9-50 |

Compound (A) potently inhibited pAKT^{S473} expression in both basal and activated cultures. The pAKT signal for healthy donors was too low to generate concentration-response data for Compound (A) reliably in the absence of activation. No significant differences in the activity of Compound (A) were observed between OKT3-stimulated or unstimulated cells, or between APDS1 or APDS2 patient-derived T lymphoblasts, by virtue of the fact that the ranges of IC50s that were obtained were overlapping.

### EXAMPLE 2: EX VIVO ANALYSIS OF PI3K SIGNALLING IN PATIENT BLOOD

### Method

The levels of pS6^{S235/236} in different T cell (CD3+CD4+; CD3+CD8+) subsets in total blood from healthy donors, and from APDS1 and APDS2 patients, were analyzed by flow cytometry. The blood was incubated ex vivo for 45 minutes at 37° C. with different doses of Compound (A) (0, 10, 30, 100, 300, 1000 and 2000 nM).

### Results

### PI3K Signalling in Lymphocytes in Whole Blood

The phosphorylation of ribosomal protein S6 at Ser 235/236 (pS6^{S235/236}) in cells from healthy donors, and from APDS1 and APDS2 patients, was analyzed ex vivo in the presence and absence of Compound (A) (10-2000 nM) in different T cell (CD3+CD4+; CD3+CD8+) subsets. As noted above, total blood was incubated ex vivo for 45 minutes at 37° C.

Data were generated with blood from two (2) healthy donors, three (3) APDS1 patients and one (1) APDS2 patient.

The results of analysis of pS6^{S235/236} expression are displayed in FIG. 3. As can be seen from FIG. 3, the expression of pS6^{S235/236} was generally elevated in the APDS1 CD3⁺ cells in total blood compared to cells from the healthy donors.

### Effect of Compound (A) on PI3K Signalling in T and B Cells in Whole Blood

In concentration-response assays, Compound (A) showed inhibition of the pS6^{S235/236} signal in three (3) T cell subsets in three (3) APDS1 patients. Representative concentration-response curves for one (1) APDS1 patient are displayed in FIG. 4. Expression of pS6^{S235/236} in the T cell subsets in blood from the healthy donors was too low to allow generation of concentration-response curves.

The IC50 values (adjusted for free concentration) of Compound (A) for pS6^{S235/236} expression in T lymphoblasts derived from three (3) APDS1 patients is summarised in Table 2:

| | CD3+ | CD8+ | CD4+ |
|---|---|---|---|
| Geomean IC50 (nM) | 51 | 56 | 41 |
| Range | 36-67 | 40-72 | 29-56 |

Data for expression of pS6^{S235/236} in T cells were available from one (1) APDS2 patient. In this patient, data from the CD3⁺ and CD8⁺ cells were reliable, whereas pS6^{S235/236} expression in CD4+ cells was too low to be reliably detected. The results are displayed in FIG. 5. As can be seen from FIG. 5, Compound (A) showed potent inhibition in this system.

### Conclusion

The level of PI3K signalling, determined by measurement of pAKT, was found to be elevated in APDS1 and APDS2 patient-derived T cell lymphoblasts. Compound (A) showed potent inhibition of pAKT expression in T cell lymphoblasts from both APDS1 and APDS2 patients. The range of IC50s achieved by Compound (A) was similar for both APDS1 and APDS2 patient-derived T cell lymphoblasts, in the absence (IC50 range: 3-20 nM) and presence (IC50 range: 7-50 nM) of T cell activation by OKT3.

In whole blood, the level of PI3K signalling, determined by measurement of pS6, was raised in T cells from the three APDS1 patients assessed, compared to healthy donors. Moreover, Compound (A) was able to inhibit expression of PI3K signalling in T cells from APDS1 patients with IC50s (adjusted for protein binding) of 51 nM (range: 36-67 nM), 56 nM (range: 40-72 nM) and 41 nM (range: 29-56 nM), for CD3⁺, CD8⁺ and CD4⁺ respectively. Data from one APDS2 patient for CD3⁺ and CD8⁺ cells were available, and showed inhibition (IC50 values) of approximately 100 nM or better, based on the concentration-response curves obtained.

In summary, Compound (A) potently inhibited PI3K signalling in APDS1 and APDS2 patient-derived cells in the same potency range, both in the presence and absence of activation by OKT3. As such, Compound (A) provides an effective treatment for individuals suffering from APDS through reversal of the hyperactivation of PI3K signalling observed in the lymphocytes of APDS patients.

### EXAMPLE 3: CLINICAL STUDY

APD001 is an ongoing Phase 1b, multicentre, open-label, 12-week study to assess the efficacy, safety and tolerability of Compound (A) in male and female adolescents (aged from 12 to 18 years) and adults with APDS1 and APDS2. Three patients have completed the 12 weeks of treatment and have displayed some clinical and immunological improvements together with disease activity improvement, as measured by the patient and the treating physician. Compound (A) was well tolerated and any Adverse Events observed did not warrant discontinuation from the study. These three patients were judged to have a positive benefit-risk balance according to the Safety Monitoring Committee of the APD001 study and therefore were enrolled in the open label extension, study APD003.

1. (canceled)

2. A method for the treatment and/or prevention of activated phosphoinositide 3-kinase delta syndrome (APDS), which method comprises administering to a patient in need of such treatment an effective amount of N—{(R)-1-[8-chloro-2-(1-oxypyridin-3-yl)-quinolin-3-yl]-2,2,2-trifluoroethyl}pyrido[3,2-d]pyrimidin-4-ylamine, or a pharmaceutically acceptable salt thereof.

3. (canceled)

4. The method according to claim 2, wherein the APDS comprises APDS1 and APDS2.

5. The method according to claim 2, wherein the APDS is APDS1.

6. The method according to claim 2, wherein the APDS is APDS2.
